(19) United States
(12) Patent Application Publication
(10) Pub. No.: US 2008/0011306 A1
(43) Pub. Date: Jan. 17, 2008

(54) Condom and Production Method Thereof

(76) Inventors: Yoshimoto Katsura, Kanagawa (JP); Hideki Gen, Kyoto (JP)

Correspondence Address: TOWNSEND & BANTA;c/o PORTFOLIO IP, PO BOX 52050, MINNEAPOLIS, MN, 55402, US

(21) Appl. No.: 11/631,669

(22) PCT Filed: Jul. 7, 2005
(86) PCT No.: PCT/JP05/12537

§ 371 Date: Sep. 23, 2007

(30) Foreign Application Priority Data
Jul. 8, 2004 (JP) 2004-201482

(51) Int. Cl. A61F 6/04

(52) U.S. Cl. 128/844

(57) ABSTRACT

[Problem to be solved] In the case where the conventional condom is used, even if it is coated with a lubricant, the surface of the penis as the male organ remains virtually kept in contact with the inside surface of the condom, and the lubricity between them is insufficient. So, since the slip that can occur between them is very slight, the physical stimulation to the penis is limited, and the condom cannot satisfy the sexual desire of the wearer.
[Solution] This invention proposes a condom 1 formed of a hydrophobic material, comprising a hydrophilic portion 3 formed on its inside surface on its tip side excluding its base side. The ratio of the length 1 of the hydrophilic portion to the overall length L of the condom excluding the small pocket formed for the semen to get collected at the tip of the condom can be set in a range of 20 to 80%, but if it is set in a range of 25 to 45%, a good balance can be achieved between the enhancement of lubricity and the effect of preventing slipping-off.

### TECHNICAL FIELD

The present invention relates to a condom and a production method thereof.

### BACKGROUND ART

A condom intended for contraception and protection from infectious diseases, especially a male condom is usually coated on the surface with a lubricant for protecting the mucous membranes and skins of the male and female organs from being injured by strong friction.

As the lubricant, for example, a jelly type silicone oil is used. The silicone oil used imparts lubricity to the surface of the condom to such an extent that too high lubricity will not cause the condom to slip off during its use.

On the other hand, the silicone oil used as a condom lubricant has such a disadvantage that if it is deposited on the clothes or the human body, it is hard to be removed. For this reason, other lubricants are proposed. For example, patent document 1 proposes a condom lubricant obtained by adding glycerol to the mixture consisting of polyvinyl pyrrolidone and a perfume, and the entire front and back surfaces of the condom material are coated with the condom lubricant.

Patent document 1: JP7-67906A

### DISCLOSURE OF THE INVENTION

### Problem to be Solved by the Invention

However, the conventional condom lubricants are used for protecting the mucous membranes and skins of the male and female organs from being injured by strong friction as described before, and not used for satisfying the sexual desire of the user, particularly of the male.

That is, in the conventional condom, even if it is coated with a lubricant, the surface of the penis as the male organ remains virtually kept in contact with the inside surface of the condom, and the lubricity between them is insufficient. So, since the slip that can occur between them is very slight, the physical stimulation to the penis is limited, and the condom cannot satisfy the sexual desire of the wearer.

For this reason, the principle that the condom should be worn from the beginning to the end of the sexual intercourse is sometimes disregarded, and it even happens that the condom is not worn at all during the sexual intercourse. So, the objects of ensuring contraception and protecting from infectious diseases are not always achieved.

On the other hand, if a condom coated on its entire front and back surfaces with a condom lubricant as disclosed in said patent document 1 is coated, for example, with a large amount of the lubricant, for enhancing lubricity, the condom may slip off during use. If the condom should slip off, the aforesaid objects of ensuring contraception and protecting from infectious diseases cannot be achieved either.

The object of this invention is to solve the aforesaid problem by providing a condom capable of assuring rationally higher lubricity, hence satisfying higher sexual satisfaction of the wearer, and yet effectively prevented from coming off.

### Means for Solving the Problem

To solve the aforesaid problem, this invention proposes a condom formed of a hydrophobic material, comprising a hydrophilic portion formed on its inside surface on its tip side excluding its base side.

Furthermore, this invention proposes the aforesaid constitution, wherein the ratio of the length of the hydrophilic portion to the overall length of the condom excluding the small pocket formed for the semen to get collected at the tip of the condom is set in a range of 20 to 80%. More suitably, the ratio of the length of the hydrophilic portion to the overall length of the condom excluding the small pocket formed for the semen to get collected at the tip of the condom is set in a range of 25 to 45%.

Still furthermore, this invention proposes the aforesaid constitution, wherein the hydrophobic material is a hydrophobic polymer such as polyurethane, polyvinyl chloride, their mixture or natural rubber latex, etc.

Still furthermore, this invention proposes the aforesaid constitution, wherein the hydrophilic portion is formed by coating the surface of the hydrophobic material with a hydrophilic material.

Still furthermore, this invention proposes the aforesaid constitution, wherein the hydrophilic material is a hydrophilic polymer or a mixture consisting of a hydrophilic polymer and a hydrophobic polymer.

Still furthermore, this invention proposes the aforesaid constitution, wherein the hydrophilic polymer is any of polyethylene glycol, polyvinyl pyrrolidone, polyacrylic acid and polyacrylamide, or any of their combinations.

Still furthermore, this invention proposes the aforesaid constitution, wherein the hydrophobic polymer as a component of the hydrophilic material is polyurethane or polyvinyl chloride or their combination.

Still furthermore, this invention proposes a method for producing a condom, comprising the steps of coating a condom formed of a hydrophobic material consisting of a hydrophobic polymer such as polyurethane, polyvinyl chloride, their mixture or natural rubber latex, with a coating solution having a hydrophilic polymer or a mixture consisting of a hydrophilic polymer and a hydrophobic polymer dissolved in an organic solvent, on the inside surface on the tip side of the condom excluding the base side; and drying to form a hydrophilic portion.

### EFFECT OF THE INVENTION

The condom of this invention has a hydrophilic portion formed on its inside surface on its tip side excluding its base side. So, when the condom is worn, the lubricity between the hydrophilic portion on the inside surface of the condom and the surface of the penis is assured.

Therefore, relative slip can occur at the portion during sexual intercourse, and the wearer can feel the same sense as he would feel without wearing the condom.

As described above, at the hydrophilic portion formed on the tip side, relative slip can occur between the inside surface of the condom and the surface of the penis, but on the inside surface on the base side of the condom, the relative slip with the surface of the penis is prevented by the frictional force of the hydrophobic material. Thus, it can be prevented that the condom slips off from the penis.

### THE BEST MODES FOR CARRYING OUT THE INVENTION

For obtaining the above effect, the ratio of the length of the hydrophilic portion to the overall length of the condom excluding the small pocket formed for the semen to get collected at the tip of the condom can be set in a range of 20 to 80%. It is more suitable to set the ratio in a range of 25 to 45%.

### EXAMPLE 1

An example of the condom of this invention will be described below.

FIG. 1 is an illustration for typically showing the condom of this invention. Symbol 1 denotes a condom formed of a hydrophobic material as described later, and 2 denotes a small pocket formed for the semen to get collected at the tip of the condom.

As described above, in this invention, the condom 1 formed of a hydrophobic material has a hydrophilic portion 3 formed on its inside surface on its tip side excluding its base side. In the drawing, symbol L indicates the overall length of the condom 1 excluding the small pocket 2 formed for the semen to get collected, and symbol 1 indicates the length of the hydrophilic portion 3.

In this invention with the aforesaid constitution, at the hydrophilic portion 3, relative slip can occur between the inside surface of the condom 1 and the surface of the penis of the wearer. Therefore, the wearer's sexual satisfaction can be significantly enhanced. On the other hand, on the inside surface on the base side of the condom 1, since the relative slip with the surface of the penis is prevented by the frictional force of the hydrophobic material, there is an effect that the condom 1 can be prevented from slipping off from the penis.

An embodiment of this invention will be described below.

At first, in the condom of this invention, the hydrophobic material is not especially limited. However, an elastic or flexible polymer such as polyurethane (PU), polyvinyl chloride (PV) or their mixture is a desirable material. An especially desirable material is polyurethane. The polyurethane can be either ester polyurethane or ether polyurethane.

As the hydrophilic material, a hydrophilic polymer can be used. For example, at least one or more compounds selected from polyethylene glycol (PEG), polyvinyl pyrrolidone (PVP), polyacrylic acid (PAA) and polyacrylamide can be used. Especially preferred is polyethylene glycol.

Furthermore, as the hydrophilic material, a mixture consisting of a hydrophilic polymer and a hydrophobic polymer can also be used. In the case where the mixture is used as the hydrophilic material, it can be familiar with the surface of the hydrophobic material of the condom 1, and can be more strongly immobilized. The hydrophilic polymer can be at least one or more compounds selected from polyethylene glycol, polyvinyl pyrrolidone, polyacrylic acid and polyacrylamide. The hydrophobic polymer can be polyurethane, polyvinyl chloride or their mixture. Especially a mixture consisting of polyethylene glycol and polyurethane is preferred.

It is desirable that the ratio by weight of the hydrophilic polymer and the hydrophobic polymer of the mixture is in a range of (hydrophobic polymer): (hydrophilic polymer)=0.1:99.9 to 90:10 (wt %). A more desirable range is (hydrophobic polymer):(hydrophilic polymer)=30:70 to 90:10 (wt %).

To obtain the aforesaid effect, the ratio (1/L) of the length 1 of the hydrophilic portion 3 to the overall length L of the condom 1 excluding the small pocket 2 formed for the semen to get collected can be set by trading off between the relative slipping possibility of the penis owing to the lubricity of the hydrophilic portion 3 and the slipping-off possibility of the condom from the penis owing to the frictional force of the hydrophobic portion. The ratio (1/L) can be set in a range of, for example, 20 to 80%, but if the ratio is set in a range of 25 to 45%, a more suitable balance can be achieved.

In the condom of this invention, the portion coated with the hydrophilic material, namely, the hydrophilic portion 3 can be further enhanced in lubricity if water is added to the portion. However, since higher lubricity could result in more likelihood of slipping off, whether or not water is added can be decided in relation with the ratio (1/L) of the length 1 of the hydrophilic portion 3 to the overall length L of the condom excluding the small pocket 2.

That is, for example, if the ratio (1/L) of the length 1 of the hydrophilic portion 3 is set at a small value to enhance the action of preventing the slipping off, adding water such as saliva to the hydrophilic portion 3 for further enhancing the lubricity of the portion is effective.

In this connection, in the condom of this invention, it is preferred that the contact angle after adding water to the hydrophilic portion 3 is 50° or less. In this case, if saliva is, for example, applied to the coating portion, the lubricity of the portion can be easily enhanced. Meanwhile, the contact angle after adding water can be measured by immersing the coating portion into distilled water and measuring by the sessile drop method using a dynamic contact angle measuring instrument (produced by Shimadzu Corp.).

The condom of this invention can be produced by molding a hydrophobic material into a condom shape, coating the aforesaid portion with a coating solution having a hydrophilic material dissolved in an organic solvent, and drying.

According to this production method, the condom of this invention can be easily produced. Furthermore, the hydrophilic material can be strongly immobilized on the surface of the condom, to form a strong hydrophilic portion.

Therefore, unlike the mere coating with a conventional jelly type lubricant such as silicone oil, it does not happen that the lubricant comes off with the lapse of time and can be held for a long period of time. Especially if the hydrophilic material is a mixture consisting of a hydrophilic polymer and a hydrophobic polymer, the immobilization is more secure.

The organic solvent used in the production method of this invention can be dichloromethane, tetrahydrofuran, acetone, methylene chloride, etc. Especially desirable is dichloromethane.

Furthermore, it is preferred that the concentration of the hydrophilic material in the coating solution is, for example, 0.05 to 5.0 wt %. More preferred is about 0.2 wt %.

Moreover, thecoatingsolutioncanbeapplied, forexample, by a method of immersing the desired portion into the solution. In this case, the immersion treatment can also be performed plural times.

### EXAMPLE 2

PEG with a molecular weight of about 2 million and ether polyurethane were dissolved into dichloromethane at any of various ratios by weight, to prepare 10 wt % solutions. The obtained solutions were cast to form complex films, and they were dried in a vacuum dryer.

Subsequently they were immersed in distilled water, and the contact angles of the complex films were measured by the sessile drop method using a dynamic contact angle measuring instrument (produced by Shimadzu Corp.). The contact angle was measured before and after immersing into distilled water. As a control, a polyurethane film was formed by casting, and the contact angle was measured similarly. The results are shown in Table 1.

TABLE 1

| PU/PEG mixing | Contact angle before | Contact angle after |
|---|---|---|
| ratio | immersion | immersion |
| Control | 71.1° | 68.6° |
| 2/3 | 62.8° | 17.8° |
| 1/1 | 67.8° | 41.4° |
| 2/1 | 62.8° | 48.8° |
| 4/1 | 72.0° | 31.9° |

As can be seen from Table 1, the complex films of the example were smaller in contact angle than the film of the control, showing higher water wettability.

### EXAMPLE 3

PEG with a molecular weight of about 2 million was dissolved into dichloromethane, to prepare 5 wt % solution. An ether polyurethane film was coated with the obtained solution and dried in a vacuum dryer. Thus, the polyurethane was coated on the surface with PEG.

Subsequently the complex film was immersed in distilled water, and the contact angle of the film was measured by the sessile drop method using a dynamic contact angle measuring instrument (produced by Shimadzu Corp.). The contact angle was measured before and after immersing into distilled water. As a control, the contact angle of a polyurethane film not coated with PEG was also similarly measured. The results are shown in Table 2.

TABLE 2

| | Contact angle before | Contact angle after |
|---|---|---|
| Sample | immersion | immersion |
| Polyurethane | 71.1° | 68.6° |
| film | | |
| PEG-coated | 66.8° | 10.9° |
| film | | |

As can be seen from Table 2, the PEG coated film was smaller in contact angle than the film not coated with PEG, showing higher water wettability.

### EXAMPLE 4

A commercially available polyurethane condom (trade name . . . Sagami Original produced by Sagami Rubber Industries Co., Ltd.; overall length excluding the small pocket formed for the semen to get collected . . . 17 cm) was put on a glass test tube (outer diameter 3 cm, length 20 cm), and in succession, it was turned inside out to cover another test tube of the same size. In this state, about 40° C. water was used to wash away the silicone oil on the inside surface, and the surface was dried using an electric fan.

Then, PEG with a molecular weight of about 2 million was dissolved into dichloromethane, to prepare 0.2 wt % solution. The tip portion of the condom turned inside out was immersed in the solution for about 2 seconds. The length of the immersed region was about 3 cm, about 5 cm or about 7 cm from the tip excluding the small pocket. After each condom was taken out of the solution, the condom was immediately dried by an electric fan, with its tip kept upward while the test tube was revolved. The tip portion deformed by immersion was returned to its original form by drying.

After sufficient drying, each condom was turned inside out again to cover another test tube, for restoring its usual style, and the base hem ring was tucked up. As a result, the silicone oil deposited on the outside surface was deposited on the inside surface of the tip portion.

Monitors were asked to use the obtained three kinds of condoms. It was found that condoms of 5 cm and 7 cm were more effective than those of 3 cm, and that the condoms of 5 cm and 7 cm were virtually equivalent to each other in effect.

Furthermore, five monitors were asked to use condoms of 5 cm. As a result, it was confirmed that when water such as saliva was added when each condom was used, the lubricity on the inner circumferential surface at the tip portion was enhanced. Furthermore, the satisfaction achieved by using the condoms was found to be far higher than that achieved by using the conventional condoms and to be equivalent to that achieved without wearing condoms. It was also found that the condoms were not likely to slip off and could be stably worn like the conventional condoms.

### INDUSTRIAL APPLICABILITY

As described above, if the condom of this invention is used, at the hydrophilic portion formed on the tip side, relative slip can occur between the inside surface of the condom and the surface of the penis of the wearer. Therefore, the sexual satisfaction felt by the wearer can be significantly enhanced, and since the relative slip between the inside surface of the condom on the base side and the surface of the penis is prevented by the frictional force of the hydrophobic material, the condom has an effect that the slipping-off of the condom from the penis can be prevented.

Therefore, since the condom of this invention can satisfy the sexual desire of the wearer, those who wear the condom of this invention will increase. So, this invention will contribute to the more widespread use of condoms. As a result, people will be able to be more thoroughly conscious of contraception, and the diffusion of sexually transmitted diseases such as AIDS will be able to be prevented. Moreover, since the condom of this invention can also be used as a substitute of female organ membrane, it can be used to deal with the sexual desire of males who do not have sex partners, serving as a measure for decreasing sexual crimes.

### BRIEF DESCRIPTION OF THE DRAWING

FIG. 1 is an illustration typically showing the condom of this invention.

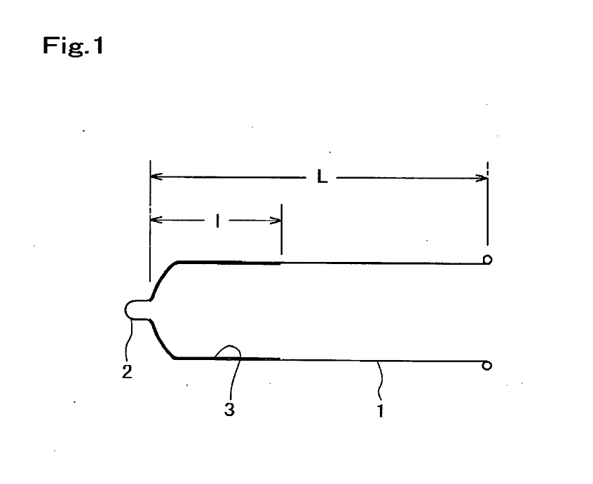

### MEANING OF SYMBOLS

1 condom
2 small pocket formed for the semen to get collected
3 hydrophilic portion

1. A condom formed of a hydrophobic material, comprising a hydrophilic portion formed on its inside surface on its tip side excluding its base side.

2. A condom, according to claim 1, wherein the ratio of the length of the hydrophilic portion to the overall length of the condom excluding the small pocket formed for the semen to get collected at the tip of the condom is set in a range of 20 to 80%.

3. A condom, according to claim 2, wherein the ratio of the length of the hydrophilic portion to the overall length of the condom excluding the small pocket formed for the semen to get collected at the tip of the condom is set in a range of 25 to 45%.

4. A condom, according to claim 1, wherein the hydrophobic material is a hydrophobic polymer such as polyurethane, polyvinyl chloride, their mixture or natural rubber latex, etc.

5. A condom, according to claim 1, wherein the hydrophilic portion is formed by coating the surface of the hydrophobic material with a hydrophilic material.

6. A condom, according to claim 5, wherein the hydrophilic material is a hydrophilic polymer.

7. A condom, according to claim 5, wherein the hydrophilic material is a mixture consisting of a hydrophilic polymer and a hydrophobic polymer.

8. A condom, according to claim 6, wherein the hydrophilic polymer is any of polyethylene glycol, polyvinyl pyrrolidone, polyacrylic acid and polyacrylamide, or any of their combinations.

9. A condom, according to claim 7, wherein the hydrophobic polymer is polyurethane or polyvinyl chloride or their combination.

10. A method for producing a condom, comprising the steps of:
coating a condom formed of the hydrophobic material as set forth in claim 4, with a coating solution comprising a hydrophilic polymer dissolved in an organic solvent, on the inside surface on the tip side of the condom excluding the base side; and
drying to form a hydrophilic portion.

11. A condom, according to claim 7, wherein the hydrophilic polymer is any of polyethylene glycol, polyvinyl pyrrolidone, polyacrylic acid and polyacrylamide,or any of their combinations.

12. A method for producing a condom; comprising the steps of:
coating a condom formed of the hydrophobic material as set forth in claim 4, with a coating solution comprising a hydrophilic material, consisting of a mixture of a hydrophilic polymer and a hydrophobic polymer, dissolved in an organic solvent, on the inside surface on the tip side of the condom excluding the base side; and
drying to form a hydrophilic portion.
